(19) United States
(12) Patent Application Publication
(10) Pub. No.: US 2019/0192716 A1
(43) Pub. Date: Jun. 27, 2019

(54) MULTIFUNCTIONAL AROMA DIFFUSER

(71) Applicant: Dalian Talent Gift Co., Ltd., Dalian (CN)

(72) Inventor: Lixin Wang, Dalian (CN)

(73) Assignee: Dalian Talent Gift Co., Ltd., Dalian (CN)

(21) Appl. No.: 16/230,948

(22) Filed: Dec. 21, 2018

(30) Foreign Application Priority Data
Dec. 22, 2017 (CN) 201711407420.1

(51) Int. Cl. A61L 9/03

(52) U.S. Cl. CPC A61L 9/03; A61L 2209/11

(57) ABSTRACT

Disclosed is a multifunctional aroma diffuser, comprising two aroma dispensing units which each can use a different type of aromatic material. The two aroma dispensing units can be used either separately or simultaneously. In one embodiment, the first aroma dispensing unit includes a platform for supporting a first aromatic material, and a lamp for activating the first aromatic material; and the second aroma dispensing unit includes a container for receiving a second aromatic material, and a heater for activating the second aromatic material. The first aromatic material can be a candle-type aromatic wax body, and the second aromatic material can be a disc-type wax pad. In another embodiment, the aroma diffuser has a base support and an alternative support for supporting aromatic materials in different sizes. The aroma diffuser also offers other functions such as supply storage, mobile charging station, and a music player.

### RELATED APPLICATIONS

The present application claims priority benefit of Chinese patent application No. CN 201711407420, filed Dec. 22, 2017, entitled “Multifunctional Aroma Diffuser, and Assembling Method Thereof”, which Chinese application is hereby incorporated in its entirety by reference.

### BACKGROUND

### Technical Field

The invention belongs to the technical field of aroma dispensing, and in particular relates to a multifunctional aroma diffuser machine.

### Background

In modern societies, people's living standards are constantly improving, and the demand for various aroma dispensing products such as aroma dispensing products is increasing. There are many aroma dispensing products in existence, especially ones that use electrically heated wax.

In a typical heat-activated aroma dispensing product, a fragrance material is solidified in a wax material, and fragrance is emitted upon heating the wax material.

But these products tend to be single-functioned and cannot satisfy multi-use purposes. For example, the user may have access to different types of aroma materials that do not all fit on a single machine. For another example, when using an aroma diffuser in a home environment, such as a living room or a kitchen, people may also want to listen to music, charge their phones, and so on.

### SUMMARY OF THE INVENTION

Disclosed is a multifunctional aroma diffuser that has different aroma diffusion mechanisms to use different types of aromatic materials. The aroma diffusion mechanisms can be used either separately or simultaneously.

It is an objective of this invention to solve the above problems by adopting a multifunctional structure that can either separately or simultaneously use multiple types of aroma materials. It is another objective of this invention to provide an aroma dispenser that can also charge a mobile phone, provide a USB interface, a storage and other functions.

In one embodiment, the aroma diffuser has two aroma dispensing units. The first aroma dispensing unit includes a platform for supporting a first aromatic material, and a lamp for activating the first aromatic material; and the second aroma dispensing unit includes a container for receiving a second aromatic material, and a heater for activating the second aromatic material.

The first aromatic material can be a candle-type wax body. A lamp mounted above the platform projects heat downward to the first aromatic material to activate the aromatic material.

The second aromatic material can be a flat disc-type wax pad, or a wax box, activated by the heater placed underneath the container.

The platform for supporting the first aromatic material can be a lower base connected to a main casing of the aroma diffuser. The lamp may have an electric heating wire. The lamp can be further controlled by a timer.

The container for receiving the second aromatic material is shaped to receive the second aromatic material in a shape of a disc, a pad or a box. The heater for activating the second aromatic material is placed under the container of the second aromatic material. The container may be made of a heat-conducting material, and the heater can be placed under the container in contact with the heat-conducting material. Likewise, the heater can be controlled by a timer.

The second aroma dispensing unit may have a cover having perforations to allow aroma diffusion when covered. The cover is preferably switchable to allow placement or replacement of the second aroma material.

The first aroma dispensing unit and the second aroma dispensing unit can be used either simultaneously or separately, each utilizing a suitable type of aroma material.

In another embodiment, the first aroma dispensing unit further has an alternative support for supporting the first aromatic material. When engaged, the alternative support is positioned above the platform such that the first aromatic material when supported by the alternative support is closer to the lamp than if supported by the platform.

An example of the alternative support has a foldable plate which, when folded up into a vertical position does not interfere the platform's supporting the first aromatic diffusion material, and when folded down into the horizontal position separates the first aromatic diffusion material from the platform.

The multifunctional aroma diffuser may further include a charging station for charging a mobile device. The charging station can be located on a top surface of the aroma diffuser to allow stable sitting of the mobile device being charged.

In another embodiment, the aroma diffuser has a multi-height support for supporting the same type of aromatic material in different sizes. For example, an alternative support is used, in addition to a primary support, for supporting the aromatic material. When engaged, the alternative support is positioned above the primary support such that the aromatic material when supported by the alternative support is closer to the lamp than if supported by the primary support. The alternative support may have a foldable plate which, when folded up into a vertical position does not interfere the platform's supporting the first aromatic diffusion material, and when folded down into the horizontal position separates and elevates the first aromatic diffusion material from the platform.

The aroma diffuser may also have other functions such as supply storage, mobile charging station, and a music player. For safety reasons, the multifunctional aroma diffuser preferably has a waterproof connector for connecting a power cord, and a non-slip rubber pad on a bottom surface.

In summary, this invention adopts a multifunctional structure that can either separately or simultaneously use an aroma wax disc, pad or box, a small lamp-melting candle-type aroma material or a large lamp-melting candle-type aroma material.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### DESCRIPTION OF DRAWINGS

The detailed description is provided with reference to the accompanying Figures. The use of the same reference numbers in different figures indicates similar or identical items.

FIG. 1 is an exploded view of a multifunctional aroma diffuser machine in accordance with the present application.

FIG. 2 is an elevational front view of the multifunctional aroma diffuser machine in accordance with the present application.

FIG. 3 is an elevational side view of the multifunctional aroma diffuser machine in accordance with the present application.

FIG. 4 is an elevational back view of the multifunctional aroma diffuser machine in accordance with the present application.

FIG. 5 is a schematic diagram of a first circuit board used in the multifunctional aroma diffuser machine to control operations.

FIG. 6 is a schematic diagram of a circuit of a music playing module used in the multifunctional aroma diffuser.

FIG. 7 is a schematic diagram of a second circuit board used in the multifunctional aroma diffuser machine to control operations.

FIG. 8 is an elevational view of the multifunctional aroma diffuser machine when the first aroma dispensing unit uses a first kind aroma source of a regular size.

FIG. 9 is an elevational view of the multifunctional aroma diffuser machine when the first aroma dispensing unit uses the first kind aroma source of a smaller size.

FIG. 10 is an elevational view of the multifunctional aroma diffuser machine showing a partial section view when the second aroma dispensing unit uses a second kind aroma source.

FIG. 11 is an elevational view of the multifunctional aroma diffuser machine showing a mobile phone being charged.

### DETAILED DESCRIPTION

Referring to the Figures, there are shown some, but not the only embodiments, of the disclosed aroma diffuser.

### Exemplary Embodiment I

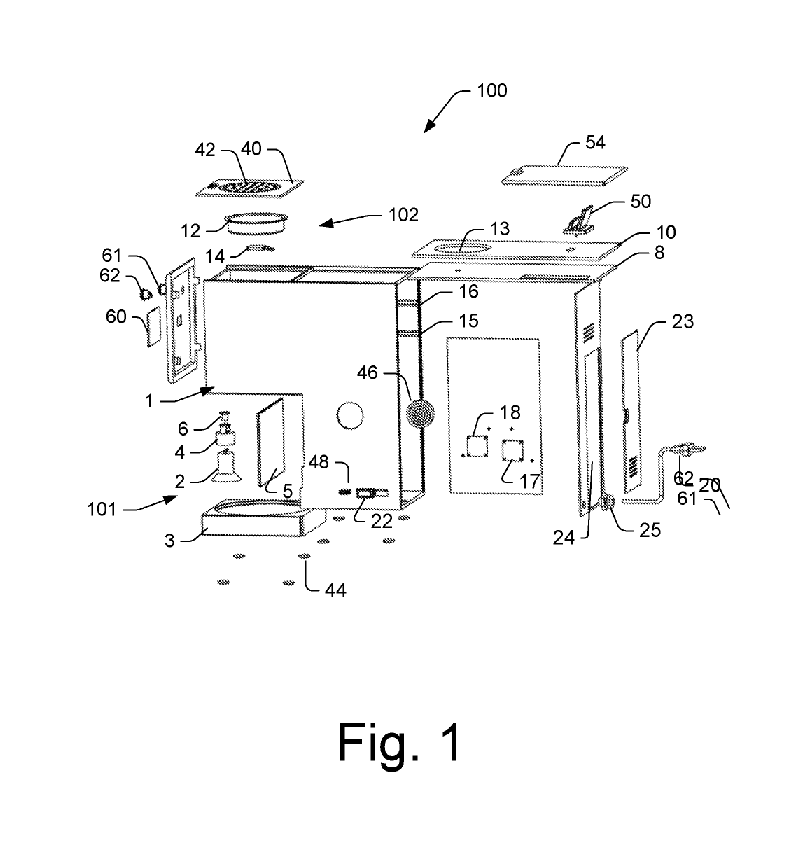

FIG. 1 is an exploded view of a multifunctional aroma diffuser machine 100 in accordance with the present application.

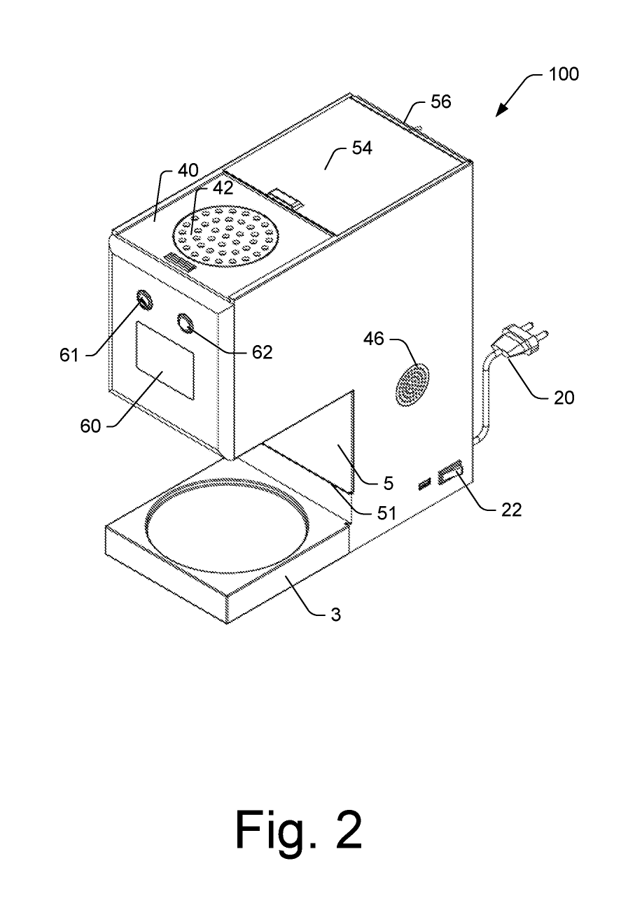

FIGS. 2-4 are several elevational views of the same multifunctional aroma diffuser 100.

As shown in FIGS. 1-4, a multifunctional aroma diffuser machine 100 includes a main casing 1, which hosts a first aroma dispensing unit 101 and a second aroma dispensing unit 102, which have different aroma dispensing mechanisms and can be used either separately or in combination.

In these embodiments, both aroma dispensing units 101 and 102 are illustrated using heat-activated aroma dispensing mechanisms, but each unit has its own characteristics.

As will be described herein, aroma dispensing units 101 and 102 each has an aroma material heating mechanism and an aroma material support mechanism.

The heating mechanism of the first aroma dispensing unit 101 includes a heating lamp 2 with a built-in heating wire (not shown), and a heating lamp holder 4, all located inside the main casing 1. The top of the heating lamp 2 is detachably connected to the heating lamp holder 4. The top of the lamb holder 4 is detachably connected through a securing nut 6 to a lamp mounting plate 8 which is also inside the main casing 1 when assembled.

The aroma material supporting mechanism of the first aroma dispensing unit 101 includes a main candle-supporting platform 3, which is connected to an outer bottom of the main casing 1 and located directly below the heating lamp 2. The connection between the candle-supporting platform 3 and the main casing 1 can be either fixed or detachable.

The aroma material supporting mechanism of the second aroma dispensing unit 102 is generally assembled on a wax tray mounting plate 10, which when assembled is disposed inside the main casing 1. The second aroma dispensing unit 102 includes a heating tray 12 and a heating plate 14. The heating tray 12 is detachably mounted on the wax tray mounting plate 10 through a matched mounting hole 13 opened on the wax tray mounting plate 10. The heating tray 12 is located directly above a heating plate 14, which can be fixedly mounted on the bottom of the heating tray 12.

The structure and the assembly of the second aroma dispensing unit 102 is better viewed in context of FIG. 1 and FIG. 10, which are an exploded view and an assembled sectional view of the unit, respectively.

The second aroma dispensing unit 102, including its heating tray 12 and the heating plate 14, is generally located above the heating lamp 2 of the first aroma dispensing unit 101.

The lamp mounting plate 8 and the wax tray mounting plate 10 and are disposed inside the main casing 1. In one embodiment, the lamp mounting plate 8 and the wax tray mounting plate 10 are detachably connected to the main casing 1 through mounting guide rails 15 and 16 on inner side walls of the main casing 1, stacking from top to bottom.

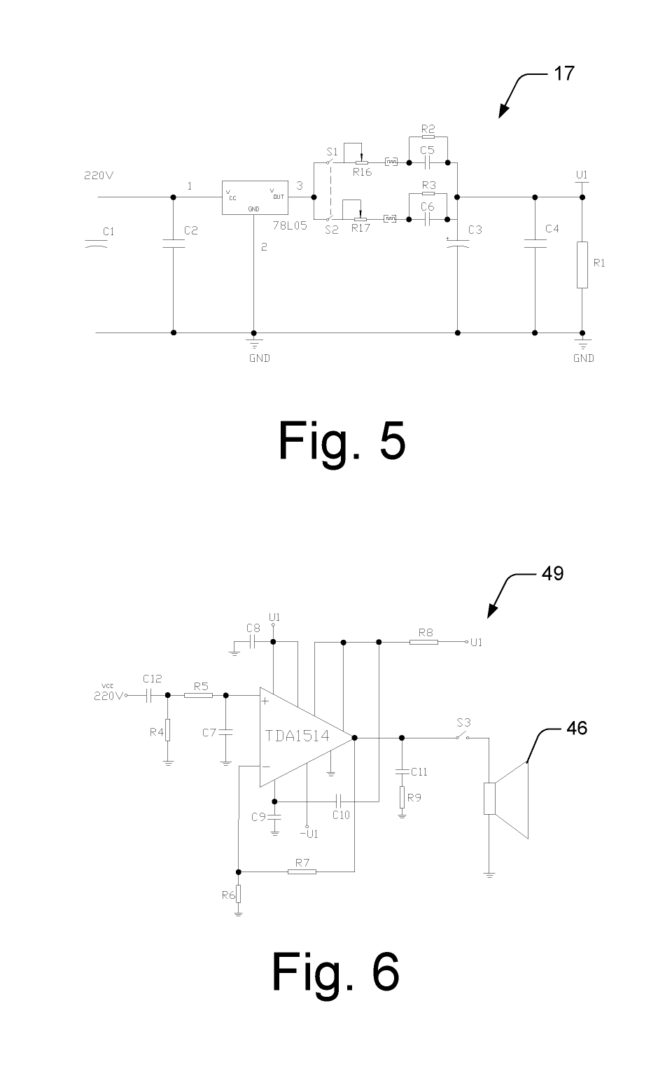

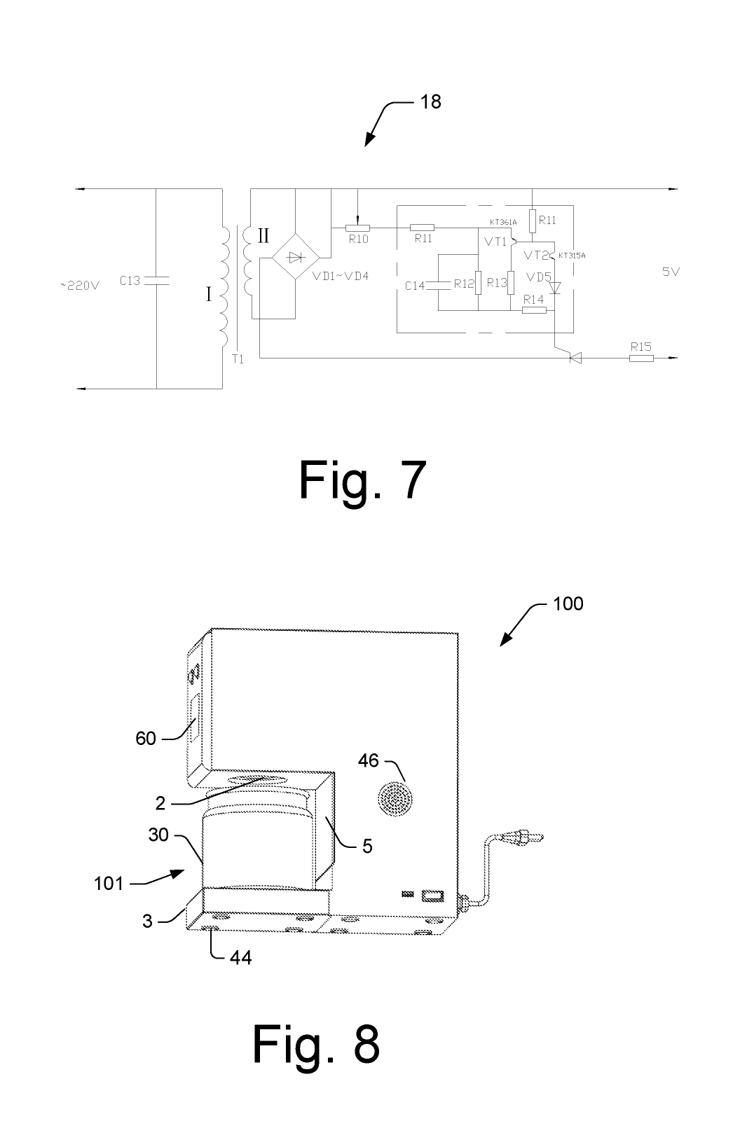

The multifunctional aroma diffuser machine 100 further has a control mechanism including a first circuit board 17 and the second circuit board 18. Exemplary embodiments of the circuit boards 17 and 18 are described in reference to FIG. 5 and FIG. 7, respectively in later sections of this description. The function of the second circuit board 18 is described in the context of another embodiment after the section.

The first circuit board 17 is for controlling the operation of the aroma diffuser machine 100, preferably through external control switches 61 and 62. In the illustrated embodiment, the first control knob 61 is for controlling the heating lamp 2, and the second control knob 62 for controlling the heating plate 14. The first control knob 61 and the second control knob 62 are mounted outside the main casing 1 through mounting slots (not shown) and detachably connected to the main casing 1 and further electrically connected to the first circuit board 17.

The first circuit board 17 is fixedly mounted inside the main casing 1, and electrically connected to a power plug 20, and further electrically connected to the heating lamp 2 and the heating plate 14.

A power switch 22 is detachably connected to the main casing 1, and electrically connected to the first circuit board 17. The power switch 22 is used to turn on the power of the multi-function aroma diffuser machine.

In order to use and control the first aroma dispensing unit 101 and the second aroma dispensing unit 102 separately, the heating lamp 2 and the heating plate 14 are preferably configured to be separately controlled by the first control knob 61 and the second control knob 62, respectively.

Preferably, the first control knob 61 and the second control knob 62 are used for respectively adjusting the heating temperature(s) of the heating lamp 2 and the heating plate 14.

The first circuit board 17 may use a chip model 78L05.

FIG. 5 is a schematic diagram of the first circuit board 17 used in the multifunctional aroma diffuser machine in accordance with the present application.

As shown in FIG. 5, after the power plug 20 is plugged in to power the multi-function aroma diffuser machine, the input voltage 220V is processed by the 78L05 chip, and the output voltage U1 required by the multi-function aroma diffuser machine is generated.

The first circuit board 17 can be configured such that the first control switch S1 (connected to the external control knob 61) and the second control switch S2 (connected to the external control knob 62) separately control the operations of the heating wires built in the heating lamp 2 and the heating plate 14. For instance, the first control switch S1 and the second control switch S2 can be provided in a manner of connection that each control switch can be opened and closed separately or simultaneously, ensuring that the heating lamp 2 of the first aroma dispensing unit 101 and the heating plate 14 of the second aroma dispensing unit 102 may work separately or simultaneously.

The heating mechanism of the first aroma dispensing unit 101 and the heating mechanism of the second aroma dispensing unit 102 can be separately controlled by adjusting varistor R16 and varistor R17 which are connected in series with the first control switch S1 and the second control switch S2, respectively. This controlling mechanism allows the heating temperature of the heating wires built in the lamp 2 and the heating temperature of the heating plate 14 to be controlled separately.

Referring back to FIGS. 1-4, in one example of the described embodiment, the aroma material support mechanism of the first aroma dispensing unit 101 further includes a foldable plate 5 serving as an alternative or ancillary aroma material support. A side of the foldable plate 5 is rotatably connected to an outside wall of the main casing 1, at a location directly above the main aroma material supporting platform 3.

Preferably, the foldable plate 5 is embedded in the main casing 1 through a rotating shaft 51.

The foldable plate 5 expands the use range of the multi-functional aroma diffuser machine. When the foldable plate 5 is rotated around the axis of the rotating shaft 51 (FIG. 2) to a vertical position, it is in a folded position and does not interfere with the main aroma material supporting platform 3. As will be shown in further detail with reference to FIG. 8, in this state, a large aroma candle can be placed on top of the main aroma supporting platform 3. The first aroma dispensing unit 101 thus performs aroma dispensing using the main aroma material supporting platform 3.

As will be shown in further detail with reference to FIG. 10, when a small melting wax type aroma wax is used, the foldable plate 5 is rotated around the axis of the rotating shaft 51 to a horizontal position, which is an unfolded position, to provide an alternative support suitable for the smaller aroma candle which may have a lesser height. When unfolded, the foldable plate 5 provides an elevated position to the smaller aroma candle for effective aroma dispensing. Essentially, the foldable plate 5 works as an alternative support for a smaller aroma candle to elevate the smaller aroma candle closer to the heating lamp 2 for more effective aroma dispensing.

Furthermore, mounting guide rails 15 and 16 allow the lamp mounting plate 8 and the wax disc mounting plate 10 to be mounted thereon respectively. The mounting guide rails 15 and 16 are mounted inside the main casing 1. The lamp mounting plate 8 and the wax disc mounting plate 10 can be more easily assembled to the main casing 1 by using the mounting guide rails 15 and 16.

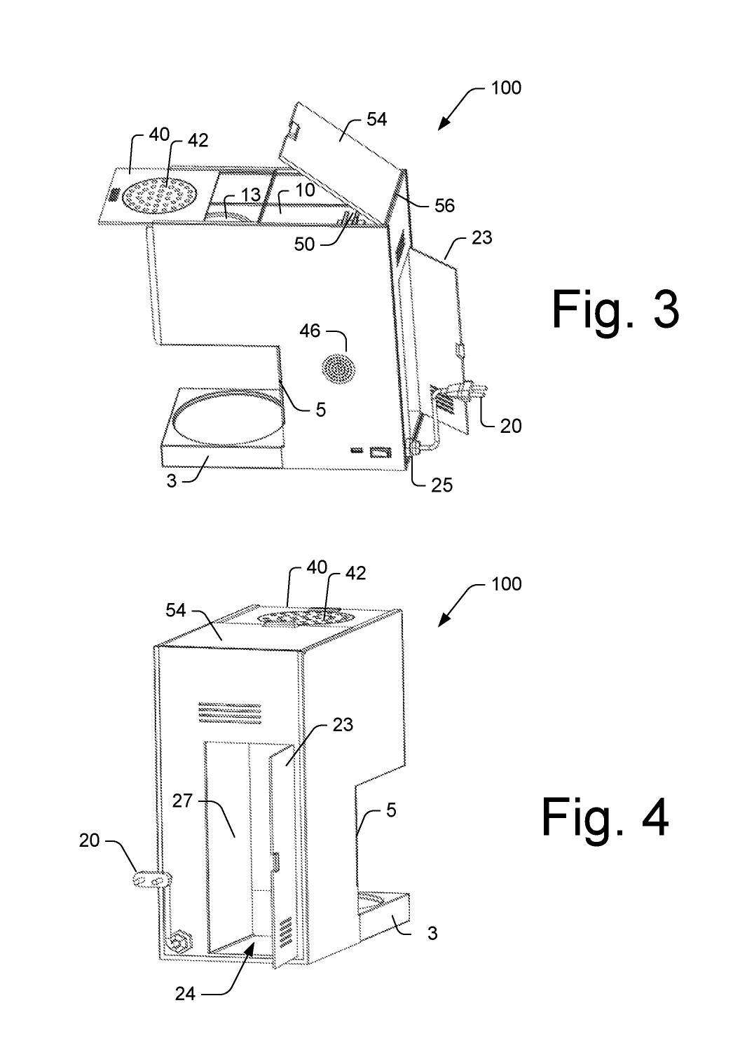

As shown in FIG. 3, in one embodiment, the main housing 1 internally forms a storage 24 for storing items such as an extra aroma material supply. The storage 24 is accessible through a storage door 23, which is in a disassemble connection with the main housing 1. Spare aroma materials can be placed inside storage 24, providing convenient access to stored aroma materials through the storage door 23.

Preferably, the first circuit board 17 is fixedly mounted on a wire protection partition 27 which separates the storage 24 from the circuit board 17 for protection of the circuit board 17.

In the exemplary embodiment described herein, the second aroma dispensing unit 102 may further include a protection plate 40, disposed above the heating tray 12. The protection plate 40 is provided with diffusing holes 42 and is detachably connected to the main casing 1.

The protection plate 40 is used to protect the heating tray 12. The diffusing holes 42 allow aroma to be more uniformly dispensed when the aroma material is heated.

Preferably, the power plug 20 is connected to the main casing 1 through a waterproof cap 25. The waterproof cap 25 seals the connection of the power plug 20 to the main casing 1.

Furthermore, the bottom of the main casing 1 is provided with non-slip rubber pads 44 attached to the bottom surface of the main casing 1, making the multi-functional aroma diffuser machine 100 more stable when placed on a flat surface.

FIGS. 8-11 show the aroma diffuser machine 100 in various operations.

FIG. 8 is an elevational view of the multifunctional aroma diffuser machine 100 when the first aroma dispensing unit 101 uses an aroma source 30 of a regular size.

An example of the aroma source 30 is a ready-made aroma candle, preferably with specs such as activation conditions and dimensions suitably made for the multifunctional aroma diffuser machine 100. A candle-shaped aroma source 30 with a suitable height can be directly placed upon the main supporting platform 3.

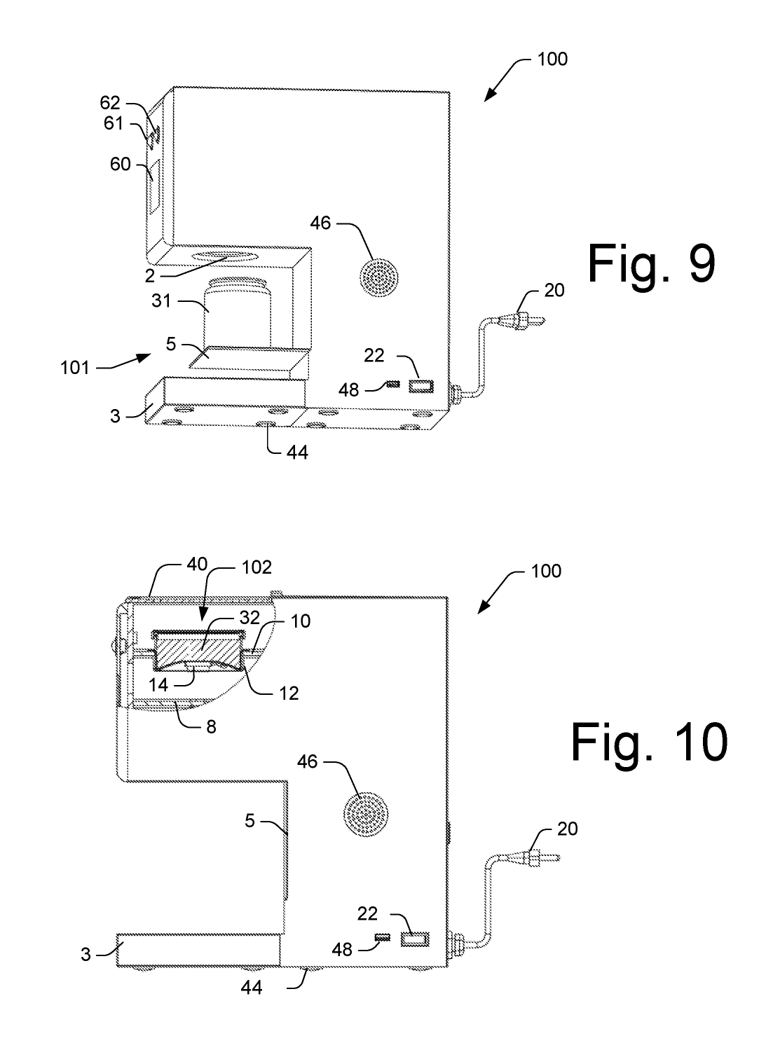

FIG. 9 is an elevational view of the multifunctional aroma diffuser machine 100 when the first aroma dispensing unit 101 uses an aroma source 31 of a smaller size.

An example of the aroma source 31 is a ready-made aroma candle, preferably with specs such as activation conditions and dimensions suitably made for the multifunctional aroma diffuser machine 100. A smaller candle-shaped aroma source 31 with a shorter height can be placed upon the alternative support foldable plate 5, rather than on the main supporting platform 3, in order to achieve a suitable distance between the top of the aroma source 31 and heating lamp 2 for effective aroma activation.

A manufacture may supply the same type of aroma candles in different sizes, for instance a larger size suited for the configuration in FIG. 8, and a smaller size suited for the configuration in FIG. 9. The customer is thus given a choice to buy and use either or both sizes of the supply.

FIG. 10 is an elevational view of the multifunctional aroma diffuser machine 102 showing a partial section view when the second aroma dispensing unit uses an alternative aroma source 32.

An example of the alternative aroma source 32 is a ready-made aroma wax disc, wax pad or wax box, preferably with specs such as activation conditions, diameter and thickness suitably made for the multifunctional aroma diffuser machine 100. Alternative aroma source 32 may come in the form of a disk or pad which can be directly placed into the heating plate 14, or may be contained in a plastic box, which can be placed into the heating tray 12. Once placed in the heating tray 12, the alternative aroma source 32 can be heated by the heating plate 14 to activate aroma dispensing. Because the wax material may melt during heating, wax in a plastic box is preferred to prevent leakage, avoid leaving residuals on the heating tray 12, and facilitate easy removal of the wax after use.

The operations shown in FIGS. 8-9 and FIG. 10 can be performed either separately or simultaneously.

Referring to FIG. 8, when turned on, the heating wire built in the heating lamp 2 is energized to heat the aroma source 30, which is a lamp-melting type aroma candle of a regular size placed on the main candle-supporting platform 3.

Referring to FIG. 9, when turned on, the heating wire built in the heating lamp 2 is energized to heat the first aroma source 31, which is a lamp-melting type aroma candle of a smaller size placed on foldable plate 3 serving as an alternative candle support.

Referring to FIG. 10, when turned on, the heating plate 14 placed under the heating tray 12 is energized to heat the second aroma source 32, which is a disc-type aroma wax contained in the heating tray 12.

During operation, aroma is released from each aroma source that is being used, either separately or simultaneously. The user can choose to use any of the illustrated aroma source alone or a suitable combination at the same time.

### Exemplary Embodiment II

The embodiment II differentiates from the embodiment I by providing a music playing module 49 (FIG. 6), with a speaker 46 and a USB interface 48. The music playing module is fixedly mounted inside the main casing 1 and is electrically connected with the power plug 20. The USB interface 48 is embedded in the main casing 1 and electrically connected to the music playing module 49. Space The speaker 46 is also electrically connected to the music playing module 49.

As shown in FIG. 6, the internal circuit 49 of the music playing module includes a TDA1514 single-chip microcomputer. The 220V voltage provided by the power source is converted into a working voltage U1 required by the music playing module after being processed by the single-chip microcomputer, and the music playing is controlled by switch S3. This enables a user to connect an electronic device such as a USB flash drive to the multifunctional aroma diffuser machine through the USB interface 48, and to play a music file in an electronic device such as a USB flash drive (not shown) by pressing a play switch (operative with the switch S3), so that the user may enjoy music while performing aroma dispensing using the aroma diffuser machine.

### Exemplary Embodiment III

The embodiment III differentiates from the embodiment I and the embodiment II by providing a timing device for controlling the operating time of the aroma diffusion machine 100 and the speaker 46. The timing device is electrically connected to the power plug 20 and the speaker 46.

Preferably, a control panel 60 is further included. The control panel 60 is detachably connected to the main casing 1 and electrically connected to the timing device.

After the multi-function aroma diffuser machine 100 is powered on by switching the power switch 22, the user can set an operating time limit by the timing device. When the multi-function aroma diffuser machine 100 has started operation, the timing device can control the speaker 46 to play a sound or music. The sound or music reminds the user that the aroma diffuser machine 100 has started to work. When the operating time reaches the limit set by the user, the timing device can control the speaker 46 to play a sound or music to remind the user that the aroma diffuser machine 100 has stopped operation. Meanwhile, the set operating time limit and the currently elapsed operating time can both be displayed on the control panel 60 to make the aroma diffuser machine more convenient to use.

### Exemplary Embodiment IV

The embodiment IV differentiates from the embodiments I, II and III by further providing a mobile phone charging stand 50. The mobile phone charging stand 50 is fixedly connected to the wax tray mounting plate 10, and has a mobile phone interface for connecting to a mobile phone 52.

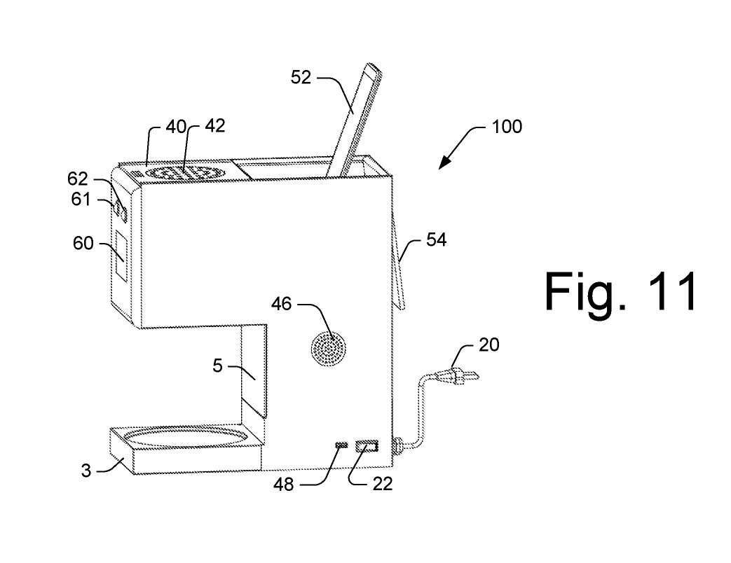

FIG. 11 is an elevational view of the multifunctional aroma diffuser machine 100 showing a mobile phone 52 being charged.

For this purpose, a second circuit board 18 (FIG. 1 and FIG. 7) is further included inside the main casing 1. The second circuit board 18 is fixedly connected to the main casing 1, electrically connected to the power plug 20, and can be further connected with mobile phone 52 through the mobile phone interface.

Furthermore, a functional flip board 54 is disposed above the mobile phone charging stand 50 and movably connected to the main casing 1.

Preferably, one end of the functional flip board 54 is embedded in the main casing 1 by connecting through a rotating shaft 56 (FIGS. 2 and 3).

When the user needs to use the charging stand 50 to charge the mobile phone 52, the functional flip board 54 can be rotated and opened along the rotating shaft 56 to reveal the mobile phone charging stand 50 for use. When finished, the flip board 54 can be rotated back to the initial position to protect the charging stand 50.

As shown in FIG. 7, the 220V AC power supplied by the power supply is converted to 36V AC through a transformer. After the rectifier circuit which includes VD1˜VD4 and R10, the AC power is converted to DC power for transmission. After being processed by the voltage divider circuit, the voltage is converted to a 5V voltage used by the mobile phone to charge. This allows the user to connect the mobile phone to the mobile phone charging stand 50 to charge the mobile phone.

According to the technical solutions described in the above embodiments, the multifunctional aroma diffuser machine 100 of the present invention can use different types of aroma sources (30, 31, and 32) either separately or simultaneously, including disc-type aroma wax pads, wax boxes, small lamp-melting type aroma candles or the large lamp-melting type aroma candles.

The machine has additional functions such as charging mobile phone, playing music and storage. The aroma diffuser described herein provides easy access to different types aroma sources, or the same type aroma source of different sizes, and potentially has reliable structure, good safety, energy saving and environmental protection with no carbon emissions nor other contaminants. The machine may have a wide range of uses and is especially suited in the home environment such as a kitchen.

Although the subject matter has been described in language specific to structural features and/or methodological acts, it is to be understood that the subject matter defined in the appended claims is not necessarily limited to the specific features or acts described. Rather, the specific features and acts are disclosed as exemplary forms of implementing the claims.

What is claimed is:

1. A multifunctional aroma diffuser comprising:
a platform which provides a base support position for supporting an aromatic material;
an alternative support which provides an alternative support position for supporting the aromatic material: and
a lamp for activating the aromatic material placed at either the base support position or the alternative support position,
wherein the alternative support position is above the base support position such that the aromatic material when supported by the alternative support is closer to the lamp than if supported by the platform.

2. The multifunctional aroma diffuser of claim 1, wherein the alternative support comprises:
a foldable plate which, when folded up into a vertical position does not interfere the platform's supporting the aromatic diffusion material, and when folded down into the horizontal position provides the alternative support position for supporting the aromatic diffusion material;

3. The multifunctional aroma diffuser of claim 1, wherein the alternative support comprises a rotatable plate embedded on a side wall of a main casing of the multifunctional aroma diffuser.

4. The multifunctional aroma diffuser of claim 1, wherein the lamp is mounted above the platform and the alternative support to project heat downward to the aromatic material when placed on either the platform or the alternative support.

5. The multifunctional aroma diffuser of claim 1, wherein the lamp has an electric heating wire.

6. The multifunctional aroma diffuser of claim 1, wherein the lamp is controlled by a timer.

7. The multifunctional aroma diffuser of claim 1, further comprising a charging station for charging a mobile device.

8. The multifunctional aroma diffuser of claim 7, the charging station is located on a top surface of the aroma diffuser to allow stable sitting of the mobile device being charged.

9. The multifunctional aroma diffuser of claim 1, further comprising a music unit for playing music.

10. The multifunctional aroma diffuser of claim 1, further comprising a storage for storing an extra aroma material.

11. The multifunctional aroma diffuser of claim 1, further comprising a waterproof connector for connecting a power cord.

12. The multifunctional aroma diffuser of claim 1, further comprising a non-slip rubber pad on a bottom surface.

13. The multifunctional aroma diffuser of claim 1, further comprising:
an additional aroma dispensing unit, including a container for receiving an additional aromatic material, and a heater for activating the additional aromatic material.

14. The multifunctional aroma diffuser of claim 13, wherein the container for receiving the second aromatic material has a shape suited for receiving the additional aromatic material shaped as a disk for pad.

15. The multifunctional aroma diffuser of claim 13, wherein the heater for activating the additional aromatic material is placed under the container.

16. The multifunctional aroma diffuser of claim 13, wherein the lamp and the heater can be used to activate the aromatic material and the additional aromatic material either separately or simultaneously.

17. The multifunctional aroma diffuser of claim 13, wherein the additional aroma dispensing unit further comprises a cover having perforations to allow aroma diffusion when covered, the cover being switchable to allow placement or replacement of the additional aroma material.

18. A multifunctional aroma diffuser comprising:
a platform which provides a base support position for supporting an aromatic material, the platform being connected to a lower portion of a main casing;
a foldable plate which, when folded up into a vertical position the foldable plate, does not interfere the platform's supporting the aromatic diffusion material, and when folded down into the horizontal position, provides an alternative support position for supporting the aromatic diffusion material; and
a lamp for activating the aromatic material placed at either the base support position or the alternative support position,
wherein the alternative support position is above the base support position such that the aromatic material when supported by the alternative support is closer to the lamp than if supported by the platform.

19. The multifunctional aroma diffuser of claim 18, further comprising:
an additional aroma dispensing unit, including a container for receiving an additional aromatic material, and a heater for activating the additional aromatic material.

20. The multifunctional aroma diffuser of claim 18, wherein the additional aroma dispensing unit is generally located above the lamp.
